(19) United States
(12) Patent Application Publication
(10) Pub. No.: US 2014/0320819 A1
(43) Pub. Date: Oct. 30, 2014

(54) SYSTEMS AND METHODS FOR DELIVERING LIGHT IN EYE TREATMENTS

(71) Applicant: Avedro, Inc, Waltham, MA (US)

(72) Inventors: David Muller, Boston, MA (US); Marc D. Friedman, Needham, MA (US)

(73) Assignee: Avedro, Inc, Waltham, MA (US)

(21) Appl. No.: 14/248,966

(22) Filed: Apr. 9, 2014

Related U.S. Application Data
(60) Provisional application No. 61/817,683, filed on Apr. 30, 2013.

(51) Int. Cl. A61B 3/00

(52) U.S. Cl. CPC A61B 3/0008
USPC 351/221

(57) ABSTRACT

A system for applying a treatment to an eye includes a housing having a first end and a second end, a contact element having an open end and a closed end, and a light source disposed within the housing and configured to direct light toward the open end. The contact element is coupled to the first end of the housing at the closed end. The open end is configured to be positioned at an eye.

### CROSS-REFERENCE TO RELATED APPLICATIONS

This application claims the benefit of priority from U.S. Provisional Application No. 61/817,683, filed Apr. 30, 2013, which is hereby incorporated by reference in its entirety.

### FIELD OF THE INVENTION

The present invention relates generally to systems and methods for treating eye disorders, and more particularly, to systems and methods that apply light during eye treatments, such as treatments that apply photosensitizing agents to cause cross-linking activity in corneal tissue.

### BACKGROUND

A variety of eye disorders, such as myopia, keratoconus, and hyperopia, involve abnormal shaping of the cornea. Laser-assisted in-situ keratomileusis (LASIK), for example, is one of a number of corrective treatments that reshape the cornea so that light traveling through the cornea is properly focused onto the retina located in the back of the eye. The success of a particular treatment in addressing abnormal shaping of the cornea depends on the stability of the changes in the corneal structure after the treatment has been applied.

Although treatments may initially achieve desired reshaping of the cornea, the desired effects of reshaping the cornea may be mitigated or reversed at least partially if the collagen fibrils within the cornea continue to change after the desired reshaping has been achieved. To strengthen and stabilize the structure of the cornea after reshaping, some treatments may also initiate cross-linking activity in the corneal tissue. For example, a photosensitizing agent (e.g., Riboflavin) is applied to the cornea as a cross-linking agent. Once the cross-linking agent has been applied to the cornea, the cross-linking agent is activated by a light source (e.g., ultraviolet (UV) light) to cause the cross-linking agent to absorb enough energy to cause the release of free oxygen radicals (e.g., singlet oxygen) and/or other radicals within the cornea. Once released, the radicals form covalent bonds between corneal collagen fibrils and thereby cause the corneal collagen fibrils to cross-link and strengthen and stabilize the structure of the cornea.

Due to the advantageous structural changes caused by the cross-linking agent, the cross-linking agent may be applied as the primary aspect of some treatments. For example, a cross-linking agent may be applied to treat keratoconus as described, for example, in European Patent Publication No. 2253321 and PCT International Patent Application Publication WO 2012/004726, the contents of these applications being incorporated entirely herein by reference.

### BRIEF DESCRIPTION OF THE DRAWINGS

FIG. 1 illustrates an example illumination device for delivering light during eye treatments, according to aspects of the present invention.

FIG. 2 illustrates another example illumination device for delivering light during eye treatments, according to aspects of the present invention.

FIG. 3 illustrates yet another example illumination device for delivering light during eye treatments, according to aspects of the present invention.

FIG. 4 illustrates a further example illumination device for delivering light during eye treatments, according to aspects of the present invention.

While the invention is susceptible to various modifications and alternative forms, a specific embodiment thereof has been shown by way of example in the drawings and will herein be described in detail. It should be understood, however, that it is not intended to limit the invention to the particular forms disclosed, but on the contrary, the intention is to cover all modifications, equivalents, and alternatives falling within the spirit of the invention.

### DESCRIPTION

Aspects of the present invention provide systems and methods that direct light to an eye during an eye treatment. In the embodiments described herein, a specially configured illumination device directs the light to corneal tissue that has been treated with a photosensitizing agent in order to initiate cross-linking activity. The illumination device delivers light according to any wavelength(s) appropriate to initiate cross-linking activity with the selected photosensitizing agent. For example, the corneal tissue may be treated with Riboflavin, and a specially configured illumination device initiates cross-linking activity by directing UV light to the treated corneal tissue. According to aspects of the present invention, the illumination device may be controlled to deliver light as a continuous wave (CW) of selected durations, etc., or as pulses according of selected durations, frequencies, etc. Additionally, the illumination device may be controlled to deliver light of selected power(s) to deliver the appropriate amount of energy to initiate cross-linking activity. For example, the illumination device may deliver light with powers of up to approximately 30 mW.

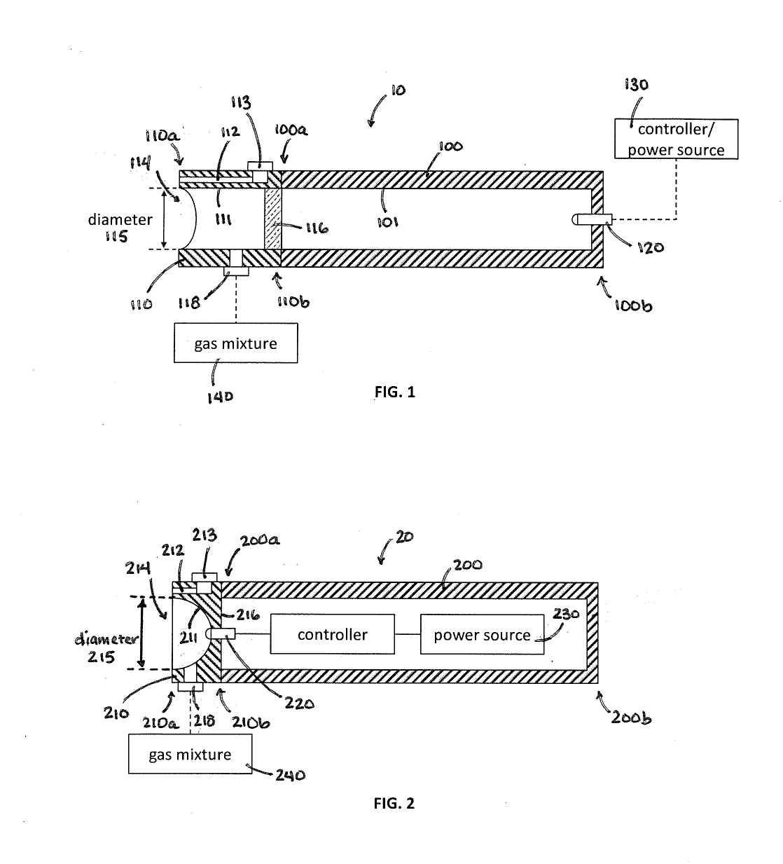

Referring to FIG. 1, an example illumination device 10 illustrates aspects of the present invention. The illumination device 10 includes a housing 100 having a first end 100a and a second end 100b. In some embodiments, the housing 100 may be a substantially cylindrical tube, but other shapes and configurations are contemplated.

The illumination device 10 also includes a contact piece 110 that is removably coupled to the housing 100 at the first end 100a. The contact piece 110 has an open end 110a which is positioned at the eye and a closed end 110b where the housing 100 is coupled. The contact piece 110 is configured to be positioned at an eye that is being treated, e.g., an eye whose corneal tissue has received a dose of cross-linking agent. The contact piece may be contoured at the open end 110a to accommodate the shape of the eye. To position the contact piece 110 stably relative to the eye, the contact piece 110 may include one or more vacuum channels 112 that are disposed along the periphery of the contact piece 110 and extend to the open end 110a. Suction is generated with the vacuum channels 112 to hold the open end 110a of the contact piece 110 against the eye. The suction may be generated, for example, by drawing air from the vacuum channels 112 with a syringe or other vacuum device coupled to the vacuum channels 112 through a vacuum port 113, which may include a valve to maintain the suction. In most embodiments, the contact piece 110 applies a pressure that is merely sufficient to position the contact piece 110 stably relative to the eye, i.e., the contact piece 110 does not apply mechanical reshaping forces to the eye.

The illumination device 10 also includes a light source 120 coupled to the housing 100 at the second end 100b. The light source 120, for example, may include light emitting diode(s) (LED(s)) from which light of selected wavelength(s), e.g., UV light, is emitted toward the contact piece 110. Any number or combination of LED's may be employed, for example, to deliver irradiances of approximately 3 mW/cm² to approximately 100 mW/cm². The light source 120 is electrically coupled to an external controller/power source system 130 that controls, and delivers power to, the light source 120. For example, the controller may cause the illumination device 10 to deliver pulsed light for a desired dose of light.

The contact piece 110 includes an opening 114 at the open end 110a through which the light from the light source 120 is directed to the eye. The opening 114 may define a substantially circular shape with a selected diameter 115, and the eye receives the light according to a similar shape. The interior walls 101 and 111 of the housing 100 and the contact piece 110, respectively, may be formed from, or coated with, a material that efficiently and effectively directs the light from the light source 120 to the eye. The material may be metallic or a diffusing white material. The interior walls, for example, may be totally reflective. As such, the illumination device 10 can deliver a homogenous beam to the eye.

Because the treatment may require light to be applied to the eye according to different patterns, e.g., circular shapes of different diameters, the contact piece 110 can be removed from the first end 100a of the housing 100 and replaced with another contact piece that has an opening defining a different pattern. For example, for one application of light, the illumination device 10 may employ a first contact piece with a substantially circular opening having a diameter 115 of approximately 9 mm, and for a subsequent application of light, the illumination device 10 may replace the first contact piece with a second contact piece with a substantially circular opening having a diameter 115 of approximately 15 mm. In some cases, the different patterns are applied to the same eye. In other cases, one pattern is applied to the eye of one subject, a different pattern is applied to the eye of another subject, and so on. In further cases, different patterns are used for different types of animals, e.g., one range of diameters is used for humans while other ranges of diameters are used for other animals (dogs, cats, horses, etc.).

The contact piece 110 may be removably coupled to the housing 100 according to any appropriate technique that allows multiple contact pieces to be coupled to the same housing 100. For example, the contact piece 110 may be coupled to the housing 100 by threaded engagement, press fit, snap fit, interlocking engagement, use of fasteners, application of adhesive, etc. The ability to change contact pieces on the same housing 100 conveniently allows the illumination device 10 to apply different patterns of light to the eye. Additionally, the ability to replace a used contact piece with a new, e.g., clean/sterile, contact piece allows the same housing 100 to be reused hygienically. A system that reuses the housing 100 with disposable contact pieces provides a cost-effective solution that reuses more costly components assembled with the housing 100, e.g., the light source 120.

To promote hygienic reuse of the housing 100, a barrier 116 is employed to prevent bacteria and other contaminants from passing into the housing 100 from the contact piece 110 which is in contact with the eye. As shown in the example of FIG. 1, the barrier 116 extends across the contact piece 110 to separate the housing 100 from the contact established with the eye. The barrier 116 is formed from a translucent material to allow the light from the light source 120 to pass from the housing 100 into the contact piece 110 and to the eye. In addition, the vacuum port 113 and a vent 118 (described below) are disposed in the contact piece 110 so that there are no passageways extending between the contact piece 110 and the housing 100, which could otherwise permit the transfer of bacteria and other contaminants to the housing 100. It is understood, however, that some embodiments may employ such passageways but may provide other protections (e.g., sterilizing steps) to promote hygienic reuse of the housing 100.

Because the light from the light source 120 travels through the barrier 116, the barrier 116 in some embodiments may include any type of optical device that modifies aspects of the light delivered to the eye. For example, the barrier 116 may include a Fresnel lens that collimates the light from the light source 120. In another example, the barrier 116 may include an optical diffuser that diffuses, spreads out, or scatters light in a desired manner. In a further example, the barrier 116 may include an array of lenslets that transmit the light according to desired focal lengths. In yet another example, the barrier 116 may include a homogenizing waveguide that transmits a homogenous beam of light.

As described, for example, in U.S. patent application Ser. No. 13/665,495, filed Oct. 31, 2012, the contents of which are incorporated entirely herein by reference, the presence of oxygen may influence the rate and amount of cross-linking activity, e.g., where singlet oxygen is generated by the application of light to the cross-linking agent. As such, the illumination device 10 also includes one or more vents that are employed to control the presence of oxygen at the eye during treatment. The one or more vents may include valves. In addition, the one or more vents may include an inlet 118 that allows oxygen to be introduced into the interior of the contact piece 110. The oxygen may be introduced via the inlet 118 from a source 140 that includes a specific concentration of oxygen to increase the amount of oxygen at the eye for cross-linking activity. The oxygen may be introduced passively by allowing the oxygen from the source 140 to diffuse into the contact piece 110 through the inlet 118. Alternatively, the oxygen may be introduced actively, e.g., as pressurized pulses, from the source 140 into the contact piece 110 through the inlet 118. In general, the inlet 118 may be employed to introduce any gas mixture to control aspects of the cross-linking activity. Additionally, the one or more vents may include an outlet (not shown) that allows gas to escape from the contact piece 110 and to maintain a particular pressure in the contact piece 110. For example, the outlet may allow the gas in the contact piece 110 and at the eye to remain at ambient pressure.

Referring to FIG. 2, another example illumination device 20 illustrates further aspects of the present invention. The illumination device 20 includes a housing 200 having a first end 200a and a second end 200b. In some embodiments, the housing 200 may be a substantially cylindrical tube, but other shapes and configurations are contemplated.

The illumination device 20 also includes a contact piece 210 removably coupled to the housing 200 at the first end 200a. The contact piece 210 has an open end 210a which is positioned at the eye and a closed end 210b where the housing 200 is coupled. The contact piece 210 includes an opening 214 at the open end 200a through which the light from the light source 220 is directed to the eye. The contact piece 210 is configured to be positioned at an eye that is being treated, e.g., an eye whose corneal tissue has received a dose of cross-linking agent. The contact piece may be contoured at the open end 210a to accommodate the shape of the eye. To position the contact piece 210 stably relative to the eye, the contact piece 210 may include one or more vacuum channels 212 that are disposed along the periphery of the contact piece 210 and extend to the open end 210a. Suction is generated with the vacuum channels 212 to hold the open end 210a of the contact piece 210 against the eye. The suction may be generated, for example, by drawing air from the vacuum channels 212 with a syringe or other vacuum device coupled to the vacuum channels 212 through a vacuum port 213, which may include a valve to maintain the suction. In most embodiments, the contact piece 210 applies a pressure that is merely sufficient to position the contact piece 210 stably relative to the eye, i.e., the contact piece does not apply mechanical reshaping forces to the eye.

The contact piece 210 also includes one or more vents that that are employed to control the presence of oxygen at the eye during treatment. The one or more vents may include valves. In addition, the one or more vents may include an inlet 218 that allows oxygen or other gas mixture from a source 240 to be introduced actively or passively into the interior of the contact piece 210 in order to control aspects of the cross-linking activity, as described above. Additionally, the one or more vents may include an outlet (not shown) that allows gas to escape from the contact piece 210 and to maintain a particular pressure in the contact piece 210. For example, the outlet may allow the gas in the contact piece 210 and at the eye to remain at ambient pressure.

The illumination device 20 also includes a light source 220. The light source 220, for example, may include LED(s) from which light of selected wavelength(s), e.g., UV light, is emitted toward the opening 214. Any number or combination of LED's may be employed, for example, to deliver irradiances of approximately 3 mW/cm² to approximately 100 mW/cm². Unlike the illumination device 10, the light source 220 is coupled to the contact piece 210, rather than the housing 200. As shown in FIG. 2, the interior wall 211 of the contact piece 210 is hemispherical or otherwise concave. The open end of the concave shape corresponds with the open end 210a of the contact piece 210. The light source 220 is disposed at the closed end 210b of the contact piece 210, at the center of the concave shape. In general, the interior wall 211 of the contact piece 210 has a shape that allows the light from the light source 220 to be directed to the open end 210a where the eye is positioned. The interior walls 211 of the contact piece 210 may be formed from, or coated with, a material that efficiently and effectively directs the light from the light source 220 to the eye. The material may be metallic or a diffusing white material. The interior walls, for example, may be totally reflective. As such, the illumination device 20 can deliver a homogenous beam to the eye.

The opening 214 defines the shape, e.g., a substantially circular pattern with a specified diameter 215, according to which the light is applied to the eye. Because the treatment may require light to be applied to the eye according to different patterns, e.g., substantially circular patterns of different diameters, the contact piece 210 can be removed from the first end 200a of the housing 200 and replaced with another contact piece that has an opening that defines a different pattern, in a manner similar to the illumination device 10 described above.

The light source 220 may be coupled with any type of optical device (not shown) that modifies aspects of the light delivered to the eye. For example, the optical device may include a Fresnel lens that collimates the light from the light source 220. In another example, the optical device may include an optical diffuser that diffuses, spreads out, or scatters light in a desired manner. In a further example, the optical device may include an array of lenslets that transmit the light according to desired focal lengths. In yet another example, the optical device may include a homogenizing waveguide that transmits a homogenous beam of light.

The light source 220 is electrically coupled to a controller/power source system 230 that controls, and delivers power to, the light source 220. The controller/power source system 230 is disposed in the housing 200 to provide a more portable solution than the illumination device 10 which is coupled to the external controller/power source system 130.

The contact piece 210 may be removably coupled to the housing 200 according to any appropriate technique that allows multiple contact pieces to be coupled to the same housing 200. For example, the contact piece 210 may be coupled to the housing 200 by threaded engagement, press fit, snap fit, interlocking engagement, use of fasteners, application of adhesive, etc. The ability to change contact pieces on the same housing 200 conveniently allows the illumination device 20 to apply different patterns of light to the eye. As described above, the light source 220 disposed in the contact piece 210. As a result, the light source 220 must also be decoupled from, or coupled to, the housing 200 when the contact piece 210 is correspondingly decoupled from, or coupled to, the housing 200. In particular, the light source 220 is decoupled from, or coupled to, the controller/power source system 230. To facilitate this action, the light source 220 may include a connector that is easily inserted into a socket for the controller/power source system 230, or vice versa.

The ability to replace a used contact piece with a new, e.g., clean/sterile, contact piece allows the same housing 200 to be reused hygienically. A system that reuses the housing 200 with disposable contact pieces provides a cost-effective solution that reuses more costly components assembled with the housing 200, e.g., the controller and power source system 230. As shown in FIG. 2, a wall 216 at the closed end 210b of the contact piece 210 prevents bacteria and other contaminants from passing from the contact piece 210 into the housing 200. As shown in the example of FIG. 2, the wall 216 extends across the contact piece 210 to separate the housing 200 from the contact established with the eye. The wall 216 is also configured to support the light source 220.

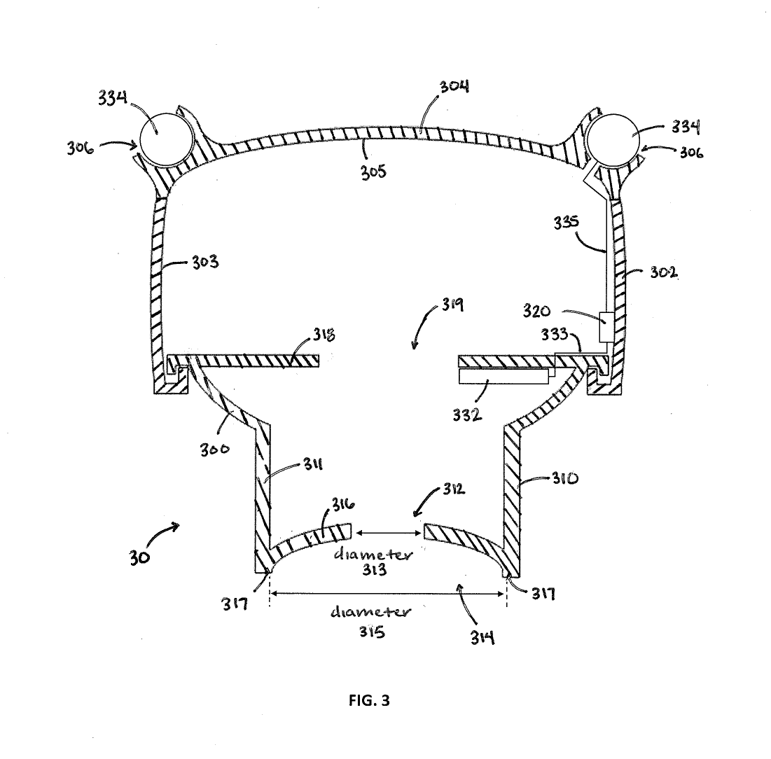

Referring to FIG. 3, yet another example of an illumination device 30 illustrates further aspects of the present invention. The illumination device 30 includes a housing 300 that is assembled from three sections 310, 302, and 304. The sections 310, 302, and 304, for example, may be molded from plastic. Unlike the housings 100 and 200 described above, the housing 300 has a generally ball-like shape. The section 310 defines a contact piece that is configured to be positioned at an eye that is being treated, e.g., an eye whose corneal tissue has received a dose of cross-linking agent. As shown in FIG. 3, the contact section 310 is coupled to the intermediate section 302 by interlocking engagement, and the intermediate section 302 is coupled to the end section 304 by welding, adhesive, snap engagement, or the like. It is contemplated that any combination of coupling techniques may be employed to assemble the sections 310, 302, and 304.

The illumination device 30 also includes a light source 320 that emits light which is directed to the contact section 310 where the eye is positioned. The light source 320, for example, may include LED(s) from which light of selected wavelength(s), e.g., UV light, is emitted. Any number or combination of LED's may be employed, for example, to deliver irradiances of approximately 3 mW/cm² to approximately 100 mW/cm².

The light source 320 is electrically coupled, e.g., by wires 333, to a controller 332 that controls the light source 320. The controller 332 may include an integrated circuit, such as a flex circuit. For example, the controller 332 may control the light source 320 to provide pulsed light for a desired dose of light. Although FIG. 3 may illustrate a particular configuration for the light source 320 and the controller 332, it is understood that the light source 320 and the controller 332 may be located in different positions within the housing 300.

The interior walls 303 and 305 of the intermediate section 302 and the end section 304 may be formed from, or coated with, a material that efficiently and effectively directs the light from the light source 320 to the contact section 310 where the eye is positioned. The material may be metallic or a diffusing white material. The interior walls, for example, may be totally reflective. As such, the illumination device 30 can deliver a homogenous beam to the eye. In general, any aspect of the interior of the housing 300 may be formed from, or coated with, a material that reflects the light from the light source 320.

Furthermore, as FIG. 3 illustrates, the housing 300 includes an internal diaphragm 318 with an aperture 319 through which the light from the light source 320 is directed to the eye. In some embodiments, an optical device of any type (not shown) may be disposed in the aperture 319 to modify aspects of the light delivered to the eye. For example, the optical device may include a Fresnel lens that collimates the light from the light source 320. In another example, the optical device may include an optical diffuser that diffuses, spreads out, or scatters light in a desired manner. In a further example, the optical device may include an array of lenslets that transmit the light according to desired focal lengths. In yet another example, the optical device may include a homogenizing waveguide that transmits a homogenous beam of light.

The light source 320 and the controller 332 are further electrically coupled to a power source 334, e.g., by wire(s) 335. The power source 334 includes one or more batteries, such as AAA cells. As shown in FIG. 3, the batteries are held in external recesses 306 formed in the end section 304. Rather than employing an on/off switch, a removable non-conducting tab (not shown) may be positioned to break the electrical connection(s) between the power source 334 and the light source 320 and/or the controller 332. The illumination device 30 is activated (“turned on”) to deliver the light by removing the non-conducting tab, which causes power to flow to the light source 320 and/or the controller 332.

The contact section 310 has a first opening 312 that is defined by an internal tab 316 that extends inwardly from the walls 311 of the section 310. In operation, the light from the light source 320 passes through the first opening 312 in a pattern defined by the first opening 312. In particular, the first opening 312 may have a substantially circular shape with a diameter 313, and as such, the light is delivered to the eye according to a similar shape.

As shown in FIG. 3, however, the internal tab 316 can be removed from the contact section 310 at a breakable connection 317. The contact section 310 has a second opening 314 that extends between the walls 311 of the contact section 310. Thus, when the internal tab 316 is removed, the light from the light source 320 passes through the second opening 314 in a pattern defined by the second opening 314, which may have a substantially circular shape with a diameter 315. Accordingly, the removable internal tab 316 allows the illumination device 30 to deliver light according to two different patterns, e.g., two different diameters. For example, for one application of light, the illumination device 30 may employ the first opening 312 to deliver light with a diameter 313 of approximately 9 mm, but for another application of light, the illumination device 30 may employ the second opening 314 with a diameter 315 of approximately 15 mm. In some cases, the different patterns may be used for different types of animals, e.g., one diameter is used for humans while another diameter is used for other animals (dogs, cats, horses, etc.). In some embodiments, multiple removable tabs may be employed to provide multiple openings, e.g., diameters, which can accommodate different uses with the single illumination device 30. In alternative embodiments, other adjustable or reconfigurable structures can be employed to allow a single illumination device to provide more than one light pattern.

Rather than using vacuum suction as described above, elastic straps (not shown) may be employed to hold the illumination device 30 at the eye. The elastic straps may fit adjustably around the head. In most embodiments, the elastic straps apply a pressure that is merely sufficient to position the illumination device 30 stably relative to the eye, i.e., the contact piece does not apply mechanical reshaping forces to the eye. Of course, the illumination device 30 may be coupled to the head according to other techniques. For example, adhesive strips may be employed.

The illumination device 30 provides a highly portable solution for delivering light for an eye treatment. The illumination device 30 integrates all necessary components in the single housing 300. Furthermore, the removable internal tab 316 allows the illumination device 30 to be configured to deliver more than one pattern of light. In an example operation, one of the patterns, e.g., diameters, of light is selected by retaining or removing the internal tab 316. Elastic straps are employed to couple the illumination device 30 to the head and the illumination device 30 is then positioned at the eye (previously treated with a cross-linking agent). The illumination device 30 is “turned on,” e.g., by removing the non-conducting tab. A desired dose of light is delivered to the eye by keeping the illumination device 30 in place for a specified amount of time. Once the amount of time has passed, the illumination device 30 is removed from the eye to end treatment (as there is no on/off switch). When the illumination device 30 is no longer required, the power source 334, i.e., batteries, can be removed from the external recesses 306 and the illumination device 30 can be discarded in an environmentally-responsible manner.

The disposable nature of the illumination device 30 is enhanced by forming the housing 300 and/or other aspects of the illumination device 30 from relatively inexpensive materials, such as plastic. However, it is understood that the embodiments described herein may be formed from any number and combination of suitable materials, including metal (e.g., aluminum), plastic, etc.

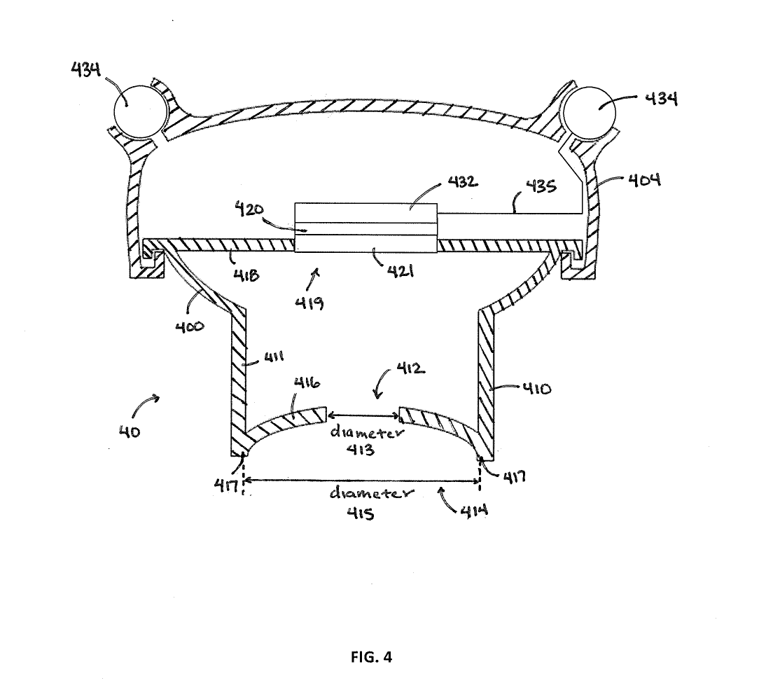

Referring to FIG. 4, a further example illumination device 40 illustrates further aspects of the present invention. The illumination device 40 includes a housing 400 that is assembled from at least two sections 410 and 404. The sections 410 and 404, for example, may be molded from plastic. The housing 400 has a generally ball-like shape like the housing 300 described above, but may be more compact due to the configuration described in more detail herein. The section 410 defines a contact piece that is configured to be positioned at an eye that is being treated, e.g., an eye whose corneal tissue has received a dose of cross-linking agent. As shown in FIG. 4, the contact section 410 is coupled to the end section 404 by interlocking engagement. It is contemplated that any combination of coupling techniques, however, may be employed to assemble the sections 410 and 404.

The illumination device 40 includes a light source 420 and a controller 432. The light source 420 emits light which is directed to the contact section 410 where the eye is positioned. The light source 420, for example, may include LED(s) from which light of selected wavelength(s), e.g., UV light, is emitted. Any number or combination of LED's may be employed, for example, to deliver irradiances of approximately 3 mW/cm² to approximately 100 mW/cm².

The controller 432 controls the light source 420. For example, the controller 432 may control the light source 420 to provide pulsed light for a desired dose of light. As shown in FIG. 4, the controller 432 may be an integrated circuit board, and the light source 420, e.g., an array of LEDs, is disposed on the side of the board that faces the eye positioned at the contact section 410.

An optical device 421 of any type may be employed to modify aspects of the light delivered to the eye from the light source 420. For example, the optical device 421 may include a Fresnel lens that collimates the light. In another example, the optical device 421 may include an optical diffuser that diffuses, spreads out, or scatters light in a desired manner. In a further example, the optical device 421 may include an array of lenslets that transmit the light according to desired focal lengths. In yet another example, the optical device 421 may include a homogenizing waveguide that transmits a homogenous beam of light.

As FIG. 4 also illustrates, the housing 400 includes an internal diaphragm 418 with an aperture 419, in which the optical device 421 is disposed. The controller 432 and the light source 420 are positioned proximate to the optical device 421, so that the light emitted from the light source 420 and controlled by the controller 432 is delivered efficiently and effectively through the optical device 421 into the contact section 410 and to the eye. In particular, the optical device 421 forms a top portion of the contact section 410, with the controller 432 disposed above the optical device 421 and the light source 420 disposed between the controller 432 and the optical device 421. In general, light from the light source 420 may be delivered more directly to the eye than light from the light source 320 described above. In some embodiments, aspects of the interior of the housing 400, e.g., interior walls of the 411 of the contact section 410, may also be formed from, or coated with, a material that helps to direct the light from the light source 420 to the eye.

The light source 420 and the controller 432 are electrically coupled to a power source 434, e.g., by wire(s) 435. The power source 434 includes one or more batteries, such as AAA cells. As shown in FIG. 4, the batteries are held in external recesses 406 formed in the end section 404. Rather than employing an on/off switch, a removable non-conducting tab (not shown) may be positioned to break the electrical connection(s) between the power source 434 and the light source 420 and/or the controller 432. The illumination device 40 is activated (“turned on”) to deliver the light by removing the non-conducting tab, which causes power to flow to the light source 420 and/or the controller 432.

Like the contact section 310 described above, the contact section 410 has a first opening 412 that is defined by an internal tab 416 that extends inwardly from the walls 411 of the section 410. In operation, the light from the light source 420 passes through the first opening 412 in a pattern defined by the first opening 412. In particular, the first opening 412 may have a substantially circular shape with a diameter 413, and as such, the light is delivered to the eye according to a similar shape.

As shown in FIG. 4, the internal tab 416 can also be removed from the contact section 410 at a breakable connection 417. The contact section 410 has a second opening 414 that extends between the walls 411 of the contact section 410. Thus, when the internal tab 416 is removed, the light from the light source 420 passes through the second opening 414 in a pattern defined by the second opening 415, which may have a substantially circular shape with a diameter 415. Accordingly, the removable internal tab 416 allows the illumination device 40 to deliver light according to two different patterns, e.g., two different diameters.

Rather than using vacuum suction as described above, elastic straps (not shown) may be employed to hold the illumination device 40 at the eye. Alternatively, the illumination device 40 may be coupled to the head according to other techniques. For example, adhesive strips may be employed.

Like the illumination device 30 described above, the illumination device 40 provides a highly portable solution for delivering light for an eye treatment. The illumination device 40 integrates all necessary components in the single housing 400. Furthermore, the removable internal tab 416 allows the illumination device 40 to be configured to deliver more than one pattern of light. In an example operation, one of the patterns, e.g., diameters, of light is selected by retaining or removing the internal tab 416. Elastic straps are employed to couple the illumination device 40 to the head and the illumination device 40 is then positioned at the eye (previously treated with a cross-linking agent). The illumination device 40 is “turned on,” e.g., by removing the non-conducting tab. A desired dose of light is delivered to the eye by keeping the illumination device 40 in place for a specified amount of time. Once the amount of time has passed, the illumination device 40 is removed from the eye to end treatment (as there is no on/off switch). When the illumination device 40 is no longer required, the power source 434, i.e., batteries, can be removed from the external recesses 406 and the illumination device 40 can be discarded in an environmentally-responsible manner. The disposable nature of the illumination device 40 is enhanced by forming the housing 400 and/or other aspects of the illumination device 40 from relatively inexpensive materials, such as plastic.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

What is claimed is:

1. A system for applying a treatment to an eye, the system comprising:
a housing having a first end and a second end;
a contact element having an open end and a closed end, the contact element being coupled to the first end of the housing at the closed end, the open end being configured to be positioned at an eye; and
a light source disposed within the housing and configured to direct light toward the open end.
